(19) United States
(12) Patent Application Publication
(10) Pub. No.: US 2025/0065314 A1
(43) Pub. Date: Feb. 27, 2025

(54) Ni IMPREGNATED METAL MODIFIED ZSM-5 CATALYST FOR DEHYDRATION OF AQUEOUS ETHANOL TO ETHYLENE AND PROCESS THEREOF

(71) Applicant: Council of Scientific & Industrial Research, New Delhi (IN)

(72) Inventors: Umesh KUMAR, Dehradun (IN); Swati -, Dehradun (IN); Akash VERMA, Dehradun (IN); Bhawna SHARMA, Dehradun (IN); Anjan RAY, Dehradun (IN)

(21) Appl. No.: 18/726,327

(22) PCT Filed: Jan. 4, 2023
(86) PCT No.: PCT/IN2023/050007

§ 371 Date: Jul. 2, 2024

(30) Foreign Application Priority Data
Jan. 6, 2022 (IN) 202211000699

(51) Int. Cl. B01J 29/46; C07C 1/24

(52) U.S. Cl. CPC B01J 29/46; C07C 1/24; C07C 2529/46

(57) ABSTRACT

The Zeolite Socony Mobil-5 (ZSM-5) catalyst is ion-exchanged with metals (Mg, Ca, Sr, Ba, Ni, Cu, Zn, La.) and impregnated with Ni to synthesize Ni/M-ZSM-5. The present invention disclosed the selective catalytic dehydration of aqueous ethanol (2-99.9 wt %) and gas-stripped ethanol into ethylene in a fixed bed continuous flow reactor at a temperature of 120-300° C. by employing a Ni/M-ZSM1-5 catalyst.

### FIELD OF THE INVENTION

The present invention relates to the synthesis of Ni impregnated metal modified ZSM-5 catalyst for the dehydration of aqueous ethanol to ethylene. More particularly, the present invention relates to the synthesis of Ni impregnated M-ZSM-5 catalysts for the dehydration of ethanol to ethylene. The present invention also relates to gas stripped ethanol dehydration process wherein ethanol is stripped using N₂/air flow, and catalytic dehydration is performed using Ni impregnated M-ZSM-5 at a temperature in the range of 120-160° C.

### BACKGROUND OF THE INVENTION

Ethene (ethylene) is the most important organic chemical, by tonnage, that is manufactured. It is the building block for a vast range of chemicals from plastics to antifreeze solutions and solvents. It also provides raw materials for various industrial chemicals, polymers, and surfactants.

Braskem, LyondellBasell, Dow Chemical Co., ExxonMobil, Chevron Phillips, Shell, and Ineos are some of the leading producers of ethylene.

Ethene (ethylene), as petrochemical is primarily produced from petroleum-derived feedstocks such as naphtha. The conventional steam cracking of naphtha generates carbon footprints; this increasing carbon footprints and environmental concern demand for the environmentally benign processes for the production of ethylene. In recent years there has been growing interest in ethanol as a feedstock for ethylene production by catalytic dehydration. Ethanol derived from the fermentation broth is diluted ethanol; if this aqueous ethanol is utilized as a feedstock for ethylene production, the cost of the azeotropic distillation can be minimized. The present study discloses a catalyst for the selective dehydration of aqueous ethanol to ethylene.

Reference may be made to U.S. Pat. Nos. 4,698,452, 4,302,357A and 4,134,926A, wherein the acidic catalysts such as ZSM-5, alumina-silica based catalysts are used for ethanol dehydration at relatively higher temperature (300-400° C.).

Reference may be made to patents WO2014171688A1, CN101747136A, wherein 95% ethanol is used for producing ethylene. The disclosed process does not utilize aqueous ethanol of less than 95% as a feedstock.

Reference may be made to U.S. Pat. No. 4,423,270, wherein the catalyst comprising of substituted phosphoric acid, in which one hydroxyl group is replaced by a hydrophobic organic group containing 4-22 carbons, is used for dehydration of aqueous ethanol (70-90%) with a conversion of 95-100% at a higher temperature range of 300-400° C.

Reference may be made to U.S. Pat. No. 4,302,357, EP2448899A2, wherein other preferred catalysts for ethanol dehydration to ethylene, such as alumina and alumina-silica, are disclosed. A catalyst called Syndol, which consists of magnesium supported on alumina, is used for the commercial production of ethylene from ethanol (N. K. Kochar, R. Merims, and A. S. Padia, Chem. Eng. Progr., June 1981, 77, 66-70).

Reference may be made to U.S. Pat. No. 4,873,392, wherein La/Ce (0.1-1 wt %) impregnated ZSM-5 (Si/Al=5-75) and triflic acid (0.5-7wt %) impregnated ZSM-5 (Si/Al=5) catalysts are reported for ethanol dehydration, which results in 95% yield of ethylene with a WHSV of 1 h⁻¹.

Reference may be made to U.S. Pat. No. 4,698,452, wherein Zn—Mn/ZSM-5 catalyst is disclosed for the dehydration of aqueous ethanol, but in the disclosed process, the dehydration was performed at a higher temperature of 400° C.

Reference may be made to WO2014171688A1, wherein La and Ga modified ZSM-5 catalyst disclosed. La-ZSM-5 (La=0.1-0.5% by weight) results in 98% conversion and selectivity, whereas Ga-ZSM-5 (Ga=0.05-1 wt %) catalyst converts 99% of ethanol with the ethylene selectivity of not less than 96% at 240° C. and a WHSV of 5 h⁻¹. The disclosed process does not utilize a feedstock having less than 95% ethanol concentration.

Reference may be made to the U.S. Pat. No. 4,670,620, wherein metal-modified silicates are disclosed. Metals having the valency of 3 and 4, mainly aluminum, beryllium, chromium, and iron, are used. Other compensating metals of group Ia, Ib, IIa, IIb, IIIa, IIIb, IVa, IVb, Va, VIb, VIIb, and VIII have also been used for the dehydration of ethanol having a concentration of 4-96% to achieve almost 100% conversion and 99% selectivity. Still, the dehydration is performed at a low LHSV (liquid hour space velocity) of 0.31 h⁻¹.

Reference may be made to U.S. Pat. Nos. 4,873,392A and 4,698,452A, wherein aqueous ethanol is used for ethylene production. U.S. Pat. No. 4,698,452A utilizes aqueous ethanol (2-19%) for the production of C₂-C₄ olefins in yield of 94% but at a comparatively higher temperature, ranging from 300-450° C. using Mn—Zn modified pentasil type zeolite catalyst.

Reference may be made to KR101504416B1, wherein dehydration catalyst containing La of 0.1 to 0.5% by weight of the ZSM-5 (Si/Al molar ratio of 20-45) at a temperature range of 220-250° C. and WHSV of 5 h⁻¹ in order to achieve 98% conversion and selectivity, utilizing 70% ethanol as a feedstock is disclosed. There is no disclosure of the use of a feedstock containing less than 70% ethanol.

Reference may be made to U.S. Pat. No. 9,266,803B2 and WO20150060259, wherein a process of only separating ethanol from fermentation broth using inert gas stripping is disclosed.

Reference may be made to an article Bioresource Technology, 137, 153-159, 2013, wherein only stripping of ethanol at a temperature of 70° C. is disclosed.

The prior art processes suffer from the drawbacks such as high reaction temperature and pressure, low catalyst durability, even some catalysts fail in regeneration after deactivation, reproducibility, and a low tolerance for the formed water during dehydration reaction. Therefore, there is a need for a simple, efficient, and economical catalyst that could be functional in the presence of high water content in feedstock and perform the conversion of ethanol derived from fermentation broth to ethylene without azeotropic distillation of fermentation broth and the necessity of high reaction temperature.

Also, the combined process of gas stripping with catalytic dehydration of gas-stripped ethanol to ethylene is a novel process for the production of ethylene from the fermentation broth. Accordingly, the present invention provides a catalyst that works efficiently for vapor phase dehydration of ethanol ranging from low concentration to absolute ethanol (2-99.9 wt %) with high ethanol conversion and ethylene selectivity. The present invention also discloses a method for the complete conversion of gas-stripped ethanol with inert gas or air for the production of ethylene with high selectivity at relatively low temperature.

### OBJECTIVE OF THE INVENTION

The main objective of the present invention is to provide the synthesis of Ni impregnated metal modified ZSM-5 catalyst for the vapor phase dehydration of aqueous ethanol (2-99.9 wt %) to ethylene with high ethanol conversion and ethylene selectivity.

Another objective of the present invention is to provide a process for the facile synthesis of catalysts, Ni/M-ZSM-5 using ion exchange and impregnation methods for the selective production of ethylene from catalytic dehydration of aqueous ethanol.

Yet another objective of the present invention is to synthesize the M-ZSM-5 catalysts, and Ni impregnated on M-ZSM-5, wherein M is selected from the metals including Mg, Ca, Sr, Ba, Ni, Cu, Zn, and La.

Still, another objective of the present invention is to provide a catalyst for the selective production of ethylene from vapor phase ethanol dehydration with high ethanol conversion (>95.0%) as well as high ethylene selectivity (>98.0%) after 100 h of reactions.

Still, another objective of the present invention is to provide a process for the dehydration of gas-stripped ethanol from aqueous ethanol solution and fermentation broth using inert gases or air for gas stripping.

### SUMMARY OF THE INVENTION

In an embodiment of the present invention, it provides a catalyst for the selective dehydration of aqueous ethanol to ethylene. More particularly, the present invention discloses the dehydration of aqueous ethanol (2-99.9 wt %) into ethylene using Ni/M-ZSM-5 catalyst.

In another embodiment of the present invention, wherein Ni impregnated metal modified ZSM-5 catalyst is synthesized. The catalyst consists of ZSM-5 with ZSM-5 with Si/Al=10-70 wt %, more preferably, 25-30 wt %, in which some of the hydrogen ions are exchanged with metal ions (M), including alkaline earth metals, transition metals, and an inner transition metal. The ion-exchanged M-ZSM-5 catalyst is impregnated with Ni to synthesize Ni/M-ZSM-5.

In another embodiment of the present invention, wherein process for the preparation of Ni/M-ZSM-5 catalyst comprises the step:
a. ion exchange of metal precursor solution with ZSM-5 consisting of Si/Al ratio in the range of 10-70 wt % and followed by drying and calcination under air at 500° C. for 5 h.
b. M-ZSM-5 obtained in step (a), is impregnated with nickel acetate (2-10 wt %) in distilled water by wet impregnation, followed by drying and calcination at 500° C. for 5 h to obtain Ni/M-ZSM-5.

In another embodiment of the present invention, a solution of the metal precursor is prepared by dissolving strontium nitrate, barium chloride, calcium chloride, magnesium nitrate, lanthanum nitrate, nickel acetate, copper nitrate, and zinc acetate in deionized water.

In another embodiment of the present invention, the synthesized catalyst is tested for the dehydration of ethanol consisting of the following steps:
a. 2 g Ni/M-ZSM-5 catalyst is sieved and loaded in a fixed bed continuous flow reactor mixed with SiC as an inert material;
b. contacting an aqueous ethanol solution in the vapor phase with the catalyst of step (a) in the presence of inert gases, including He, Ar, and N₂. The ethanol-water solution, containing 2-99.9 wt % ethanol, is first vaporized and passed over the catalyst obtained in step (a) in the fixed bed reactor;
c. dehydration reaction at the temperature of 120-300° C., with the feed WHSV varying from 3-30 h⁻¹, whereas the carrier gas flow ranges from 1-100 mL min⁻¹ to achieve high ethanol conversion with high ethylene selectivity.

### BRIEF DESCRIPTION OF DRAWINGS

The present invention is illustrated in FIGS. 1 to 3 of the drawings accompanying this specification. In the drawings like reference numbers/letters indicate corresponding parts in the various figures.

FIG. 1 represents the effect on conversion and selectivity with reaction time

FIG. 2 represents the XRD pattern of (a) ZSM-5 (b) Ba-ZSM-5 (c) Mg-ZSM-5 (d) Ca-ZSM-5 (e) Sr-ZSM-5 (f) Ni/Ba-ZSM-5 (g) Ni/Mg-ZSM-5 (h) Ni/Ca-ZSM-5 (i) Ni/Sr-ZSM-5

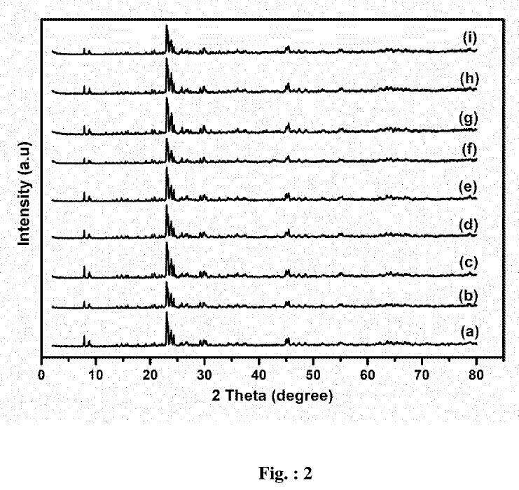

FIG. 3 represents the XRD pattern of (a) ZSM-5 (b) Ni-ZSM-5 (c) Cu-ZSM-5 (d) Zn-ZSM-5 (e) La-ZSM-5 (f) Ni/Ni-ZSM-5 (g) Ni/Cu-ZSM-5 (h) Ni/Zn-ZSM-5 (i) Ni/La-ZSM-5

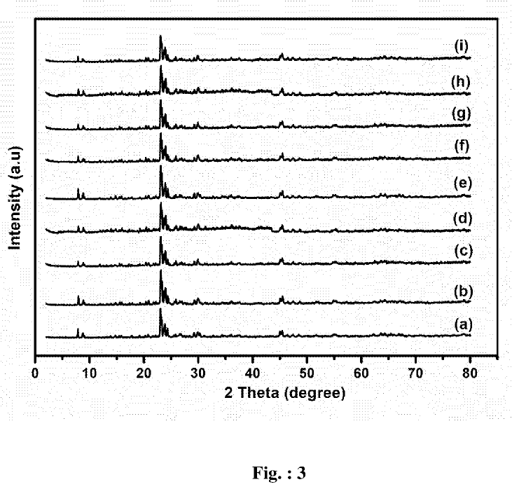

### DETAILED DESCRIPTION OF THE INVENTION

While the invention has been disclosed with reference to certain embodiments, it will be understood by those skilled in the art that various changes may be made, and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein. In line with the above objectives,

[symbol]

The present invention provides Ni impregnated metal modified ZSM-5 catalyst for the dehydration of aqueous ethanol to ethylene. The present invention relates to the synthesis of a catalyst effective for converting aqueous ethanol (2-99.9 wt %) into ethylene. The catalyst comprises metal incorporated ZSM-5. Metal ions (M) are incorporated by ion exchange, followed by the incorporation of Ni using the wet impregnation method.

In another embodiment of the present invention, the Bronsted acidity of H-ZSM-5 is controlled by exchanging the hydrogen ion present in H-ZSM-5 with other metal ions, including Mg, Ca, Sr, Ba, Ni, Cu, Zn, and La. The present invention also provides a method for the synthesis of M-ZSM-5, wherein the suspension of ZSM-5 is added to the solution of metal precursor and stirred for 12 h. Thereafter, the solution is washed thoroughly with water and dried, followed by calcination under air at 500° C. for 5 h. Ni is impregnated over M-ZSM-5 by wet impregnation. In this method, the nickel acetate solution in water is poured over ZSM-5 and allowed to dry at room temperature, followed by calcination at 500° C. for 5 h to synthesize Ni/M-ZSM-5.

The present invention also provides a catalyst for the dehydration of aqueous ethanol to ethylene. The process comprising the sequential steps of
a. providing the catalyst in 35 mesh size
b. providing the providing the feed at a WHSV of 3-30 h⁻¹
c. providing the reaction temperature of 120-300° C.
d. feed in which the ethanol concentration in feed ranges from 2-99.9 wt %

In an embodiment of the present invention, catalytic dehydration is also carried out for the gas-stripped ethanol at a temperature range of 120-160° C. In a detailed process, the gas is passed through the aqueous solution or fermentation broth of ethanol kept at RT to 45° C.; the gas carrying the ethanol vapor from the solution is passed through the reactor, where the dehydration occurs at a temperature range of 120-160° C.

The work describes the development of a novel Ni/M-ZSM-5 catalytic system suitable for the dehydration of aqueous ethanol to ethylene. The catalyst exhibits stability in activity performance for at least up to 100 h with 95.0% ethanol conversion and 98.0% ethylene selectivity.

In an embodiment of the present invention, the conversion of gas-stripped ethanol is >99%, and ethylene selectivity is >97%.

### EXAMPLES

The following examples, which include preferred embodiments, will serve to illustrate the practice of this invention, it being understood that the particulars shown are by way of example and for the purpose of illustrative discussion of preferred embodiments of the invention.

### Example 1

The example illustrates the preparation of catalysts of the invention. M-ZSM-5 is prepared by modifying H-ZSM-5 by ion-exchange method followed by drying and calcination where M=Sr, Ba, Ca, Mg, Ni, Zn, Cu, and La. Strontium nitrate, barium chloride, calcium chloride, magnesium nitrate, lanthanum nitrate, nickel acetate, copper nitrate, and zinc acetate are the metal precursors used for the preparation of catalysts.

In order to prepare M-ZSM-5 (0.2-5 wt %), a solution of metal precursors in deionized water is mixed with the suspension of ZSM-5 in 210 mL and stirred for 12 h at 70° C.; the solution is then centrifuged and washed thoroughly with deionized water until the free ions are removed. The obtained solid is then dried overnight in an air oven at 70° C., followed by calcination under an air atmosphere at 500° C. for 5 h.

Prepared catalysts, M-ZSM-5, are further modified by Ni incorporation. Ni is incorporated by the wet impregnation method. M-ZSM-5 is brought in contact with an aqueous solution of nickel acetate (2-10 wt %), dried overnight in an air oven at 70° C., followed by calcination under an air atmosphere at 500° C. for 5 h.

The performance evaluation of the M-ZSM-5 and Ni/M-ZSM-5 catalysts in the present invention is carried out in a continuous flow fixed bed reactor in the vapor phase under atmospheric pressure conditions. The reactor is equipped with a digital temperature controller that maintains the required temperature of the catalyst bed. The reactor is also equipped with a mass flow controller to control the flow rate of gases used in the reaction. The product gas stream from the reactor outlet goes through a liquid and gas separator, where gas and liquid product streams undergo separation. The liquid products are then collected in a vessel, while the gas stream is analyzed by online gas chromatography (GC). The product streams are analyzed to calculate the conversion of ethanol and the selectivity for produced ethylene.

In a detailed procedure, 2 g of the catalyst (35 mesh size, 420 μm) is loaded in the reactor with inert SiC (used as a diluent). The loaded reactor is heated and maintained at the required temperature by a digital temperature controller. Prior to the reaction, the activation of the catalyst is carried out under a continuous flow of N₂ at atmospheric pressure and a temperature of 450°° C. for a period of 3 h. The catalyst Ni/M-ZSM-5 is reduced under hydrogen at 350° C. for 2 h prior to the reaction. After the activation, the catalyst is tested for the selective dehydration of ethanol into ethylene for a minimum reaction time of 5 h. The ethanol dehydration is performed at different reaction conditions, including a temperature range of 120-300° C., WHSV of 3-30 h⁻¹, and carrier gas flow of 1-100 mL min⁻¹.

The analysis of the product is done by GC, typically equipped with a flame ionization detector (FID) and thermal conductivity detector (TCD) having HP-PLOT Q column (length 30 m, internal diameter 530 μm) connected to FID for hydrocarbons analysis (C₁-C₄) and Shin carbon column (length 2 m, inner diameter of 2 mm) connected to TCD. The ethanol conversion and ethylene selectivity (among gaseous hydrocarbons) are calculated as follows.

$$X = \frac{moles of ethanol fed into the reactor - moles of ethanol left after reaction}{moles of ethanol fed into the reactor} \times 100$$

$$S = \frac{mole of ethylene produced}{moles of total gaseous product produced} \times 100$$

X=Ethanol conversion
S=Ethylene selectivity

### Example 2

The example illustrates the optimization of the reaction parameters for ethanol dehydration. To optimize the reaction parameters, the reactions are performed at a temperature range of 120-300° C., WHSV range of 3-30 h⁻¹, and carrier gas flow of 1-100 mL min⁻¹.

The effect of temperature, WHSV, and carrier gas flow is shown in Tables 1, 2, and 3, respectively.

TABLE 1
Effect of temperature on conversion and selectivity of ethylene

| Temperature (° C.) | Conversion (%) | Selectivity (%) |
|---|---|---|
| 120 | 30.0 | 100.0 |
| 190 | 61.9 | 99.7 |
| 230 | 79.0 | 99.3 |
| 250 | 99.0 | 99.6 |
| 270 | 99.8 | 99.0 |
| 300 | 99.5 | 99.2 |

Reaction conditions: Conc. of ethanol = 6%, WHSV = 6 h⁻¹, Carrier gas flow = 20 mL min⁻¹, Time = 5 h.

TABLE 2
Effect of WHSV on conversion and selectivity of ethylene

| WHSV (h⁻¹) | Conversion (%) | Selectivity (%) |
|---|---|---|
| 3 | 99.2 | 99.2 |
| 6 | 99.5 | 99.6 |
| 9 | 99.2 | 99.7 |
| 12 | 98.6 | 99.7 |
| 18 | 97.8 | 99.7 |
| 30 | 95.1 | 99.9 |

Reaction conditions:
Conc. of ethanol = 6%,
Temperature = 250° C.,
Carrier gas flow = 20 mL min⁻¹,
Time = 5 h

TABLE 3
Effect of carrier gas flow on conversion and selectivity of ethylene

| Carrier gas flow | | |
|---|---|---|
| (mL min⁻¹) | Conversion (%) | Selectivity (%) |
| 100 | 98.7 | 98.0 |
| 70 | 98.9 | 98.2 |
| 50 | 98.8 | 98.5 |
| 30 | 99.0 | 99.5 |
| 20 | 99.2 | 99.5 |
| 10 | 99.5 | 99.5 |
| 1 | 99.1 | 99.3 |

Reaction conditions:
Conc. of ethanol = 6%,
Temperature = 250° C.,
WHSV = 9 h⁻¹,
Time = 5 h

### Example 3

The example illustrates the effect of the concentration of ethanol on conversion and ethylene selectivity. At optimized reaction conditions, i.e., temperature=250° C., WHSV=9 h⁻¹, and carrier gas flow=10 mL min⁻¹, the catalyst is also tested for dehydration of feedstock of varying ethanol concentration ranging from 2-99.9 wt % (Table 4).

TABLE 4
Effect of concentration of ethanol on
conversion and selectivity of ethylene

| Ethanol | | | Carrier | | |
|---|---|---|---|---|---|
| Conc. | Temp. | WHSV | gas flow | Conversion | Selectivity |
| (wt %) | (° C.) | (h⁻¹) | (mL min⁻¹) | (%) | (%) |
| 2 | 250 | 9 | 10 | 99.9 | 99.8 |
| 6 | 250 | 9 | 10 | 99.5 | 99.5 |
| 10 | 250 | 9 | 10 | 99.0 | 99.5 |
| 30 | 250 | 9 | 10 | 97.9 | 99.4 |
| 50 | 250 | 9 | 10 | 93.3 | 99.4 |
| 70 | 250 | 9 | 10 | 83.7 | 98.9 |
| 95 | 250 | 9 | 10 | 66.4 | 98.9 |
| 99.9 | 250 | 9 | 10 | 56.0 | 99.0 |

Reaction conditions:
Temperature = 250° C.,
WHSV = 9 h⁻¹,
Carrier gas flow = 10 mL min⁻¹,
Time = 5 h

### Example 4

The example illustrates the performance of various synthesized catalysts. All the prepared catalysts are tested at the optimized reaction conditions (Table 5).

TABLE 5
Effect of different catalysts on conversion and selectivity
of ethylene at optimized reaction conditions

| | Conversion/ | Time (h) | | | | |
|---|---|---|---|---|---|---|
| Catalysts | Selectivity (%) | 1 h | 2 h | 3 h | 4 h | 5 h |
| Mg-ZSM-5 | Conversion | 98.9 | 98.9 | 98.6 | 98.3 | 98.3 |
| | Selectivity | 100 | 100 | 100 | 100 | 100 |
| Ni/Mg-ZSM-5 | Conversion | 98.3 | 98.0 | 97.0 | 96.0 | 94.2 |
| | Selectivity | 97.4 | 98.0 | 99.0 | 99.0 | 99.6 |
| Ca-ZSM-5 | Conversion | 99.6 | 99.2 | 99.7 | 99.6 | 99.5 |
| | Selectivity | 100 | 100 | 100 | 100 | 100 |
| Ni/Ca-ZSM-5 | Conversion | 99.6 | 99.6 | 99.6 | 99.6 | 99.8 |
| | Selectivity | 98.8 | 99.7 | 99.8 | 99.8 | 99.9 |
| Sr-ZSM-5 | Conversion | 99.5 | 98.0 | 98.4 | 98.6 | 98.5 |
| | Selectivity | 99.6 | 99.7 | 99.8 | 99.8 | 99.9 |
| Ni/Sr-ZSM-5 | Conversion | 99.0 | 99.0 | 99.1 | 99.4 | 99.5 |
| | Selectivity | 98.6 | 99.0 | 99.5 | 99.5 | 99.5 |
| Ni-ZSM-5 | Conversion | 99.7 | 99.6 | 99.6 | 99.6 | 99.6 |
| | Selectivity | 99.8 | 99.8 | 99.9 | 99.99 | 99.9 |
| Ni/Ni-ZSM-5 | Conversion | 98.5 | 99.0 | 99.2 | 99.2 | 99.4 |
| | Selectivity | 98.5 | 99.0 | 99.2 | 99.5 | 99.6 |
| Cu-ZSM-5 | Conversion | 100 | 100 | 100 | 100 | 100 |
| | Selectivity | 99.9 | 99.4 | 99.4 | 99.5 | 99.5 |
| Ni/Cu-ZSM-5 | Conversion | 99.5 | 98.0 | 96.0 | 95.0 | 89.9 |
| | Selectivity | 99.6 | 99.6 | 99.5 | 99.8 | 99.7 |
| Zn-ZSM-5 | Conversion | 85.0 | 76.2 | 76.7 | 76.7 | 76.7 |
| | Selectivity | 100.0 | 99.9 | 99.9 | 99.9 | 99.9 |
| Ni/Zn-ZSM-5 | Conversion | 98.3 | 99.0 | 99.2 | 99.0 | 99.0 |
| | Selectivity | 99.7 | 99.0 | 99.0 | 99.2 | 98.0 |
| La-ZSM-5 | Conversion | 98.4 | 99.0 | 99.1 | 99.2 | 99.1 |
| | Selectivity | 99.8 | 99.8 | 99.9 | 99.9 | 99.9 |
| Ni/La-ZSM-5 | Conversion | 99.3 | 99.0 | 99.1 | 98.9 | 98.8 |
| | Selectivity | 100 | 100 | 100 | 100 | 100 |

Reaction conditions: Conc. of ethanol = 6%, Temperature = 250° C., WHSV = 9 h⁻¹, Carrier gas flow = 10 mL min⁻¹, TOS = 5 h

### Example 5

The example illustrates the performance of Ni/Ba-ZSM-5 catalyst for ethanol dehydration reaction at a temperature range of 190-250° C. with varying WHSV (Table 6).

TABLE 6
Dehydration of ethanol with Ni/Ba-ZSM-5 at
different reaction temperatures and WHSV

| Temperature | WHSV | Gas flow rate | Conversion | Selectivity |
|---|---|---|---|---|
| (° C.) | (h⁻¹) | (mL min⁻¹) | (%) | (%) |
| 190 | 6 | 20 | 94.2 | 99.4 |
| 200 | 6 | 20 | 99.2 | 99.5 |
| 200 | 9 | 20 | 86.8 | 99.9 |
| 200 | 12 | 20 | 83.2 | 99.9 |
| 220 | 6 | 20 | 99.2 | 99.2 |
| 230 | 6 | 20 | 99.6 | 99.0 |
| 250 | 6 | 20 | 99.8 | 98.6 |
| 250 | 6 | 20 | 97.4 | 96.9 |

### Example 6

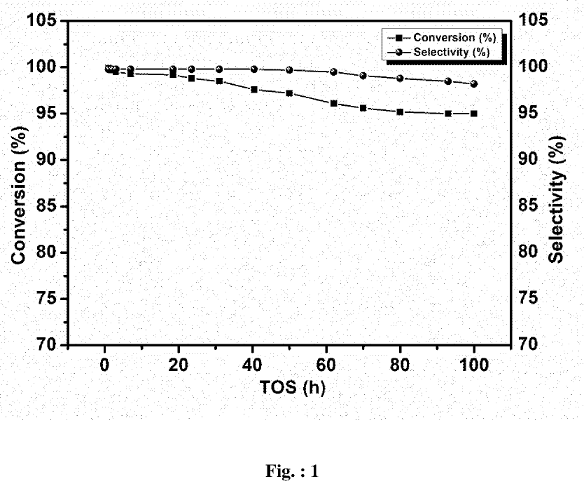

The example illustrates the activity of the catalyst Ni/Sr-ZSM-5 for 100 h. The conversion after 100 h is 95.0%, and the ethylene selectivity is 98.0% at the following reaction conditions: Conc. of ethanol=6%, Temperature=250° C., WHSV=9 h⁻¹, Carrier gas flow=10 mL min⁻¹, TOS=100 h (FIG. 1).

### Example 7

The example illustrates the catalytic dehydration by the gas stripping method using N₂/air at a temperature range of 120-160° C. using Ni/Ca-ZSM-5. In a detailed process, the gas is passed through the aqueous solution of ethanol kept at RT to 45° C.; the gas carrying the ethanol undergoes dehydration at a temperature range of 120-160° C. (Table 7).

TABLE 7
Dehydration of gas-stripped ethanol

| | | | Gas flow | Selectivity of | Selectivity of |
|---|---|---|---|---|---|
| | | Temp | rate | ethylene | diethyl ether |
| Feed | Gas | (° C.) | (mL min⁻¹) | (%) | (%) |
| 6% | N₂ | 160 | 10 | 65.3 | 34.3 |
| ethanol | | | | | |
| 6% | N₂ | 160 | 20 | 91.3 | 8.6 |
| ethanol | | | | | |
| 6% | N₂ | 160 | 30 | 97.5 | 2.5 |
| ethanol | | | | | |
| 6% | N₂ | 160 | 50 | 98.0 | 2.0 |
| ethanol | | | | | |
| 6% | N₂ | 140 | 50 | 17.3 | 82.6 |
| ethanol | | | | | |
| 6% | N₂ | 120 | 50 | 16.9 | 83.0 |
| ethanol | | | | | |
| Broth | N₂ | 160 | 50 | 97.8 | 2.2 |
| Broth | Air | 160 | 50 | 97.5 | 2.5 |

### Advantages of the Invention

The several advantages of the present process are given below;
1. The synthetic process of Ni/M-ZSM-5 catalyst used for the dehydration of ethanol into ethylene is economical, straightforward, and has a higher turnover number, which could be more favorable for the industry.
2. Aqueous ethanol (2-99.9 wt %) can be dehydrated to ethylene using the Ni/M-ZSM-5 catalyst at a relatively high WHSV (3-30 h⁻¹).
3. Gas stripping of ethanol from ethanol solution and fermented broth using N₂/air as stripping gas.
4. Gas-stripped ethanol can be dehydrated to ethylene using the Ni/M-ZSM-5 catalyst at a relatively low temperature, i.e., 160° C.

1. A catalytic dehydration process of aqueous ethanol to ethylene in vapor phase in a fixed bed reactor using Ni impregnated metal modified ZSM-5 comprising the steps of:
(a) pelletizing, crushing, sieving, and loading the Ni/M-ZSM-5 catalyst in a fixed bed continuous flow reactor mixed with a SiC as an inert material;
(b) contacting aqueous ethanol solution in vapor phase with the catalyst of step (a) in presence of inert gases, including He, Ar, N₂;
(c) vaporizing an aqueous ethanol solution and passed over the catalyst obtained in step (a) in the fixed bed reactor; and
(d) dehydrating ethanol at a temperature of 120-300° C., with the flow rate of the ethanol-water mixture varying from 3-30 h⁻¹, whereas the carrier gas flow ranges from 1-100 mL min⁻¹ to achieve ethanol conversion with high ethylene selectivity.

2. The process as claimed in claim 1, wherein ethanol is 2-99.9 wt %.

3. The process as claimed in claim 1, wherein ethanol from the solution of aqueous ethanol/fermentation broth is stripped with N₂/air and dehydration is carried out at a temperature ranging from 120-160° C., at atmospheric pressure with 98% ethylene selectivity.

4. The process as claimed in claim 1, wherein Ni/M-ZSM-5 is active at least up to 100 h with the ethanol conversion of >95.0% and ethylene selectivity of >98.0%.

5. A process for preparation of Ni impregnated metal modified ZSM-5 catalyst for catalytic dehydration of aqueous ethanol to ethylene in vapor phase, the process comprising the step of:
(a) ion exchange of metal precursor solution with ZSM-5 consisting of Si/Al ratio in the range of 10-70 wt % and followed by drying and calcination under air at 500° C. for 5 h; and
(b) impregnating Ni (2-10 wt %) over M-ZSM-5 obtained in step (a) in distilled water by wet impregnation, followed by drying and calcination at 500°° C. for 5 h to obtain Ni/M-ZSM-5.

6. The process as claimed in claim 5, wherein the solution of metal precursors is selected from the group consisting of strontium nitrate, barium chloride, calcium chloride, magnesium nitrate, lanthanum nitrate, nickel acetate, copper nitrate, and zinc acetate.

7. The process as claimed in claim 6, wherein nickel acetate is used as a precursor of nickel for impregnation.
